(19) United States
(12) Patent Application Publication
(10) Pub. No.: US 2021/0283011 A1
(43) Pub. Date: Sep. 16, 2021

(54) Wellness Device

(71) Applicant: Gary Hailey, Pipestone, MN (US)

(72) Inventor: Gary Hailey, Pipestone, MN (US)

(21) Appl. No.: 17/193,282

(22) Filed: Mar. 5, 2021

Related U.S. Application Data
(60) Provisional application No. 62/988,457, filed on Mar. 12, 2020.

(51) Int. Cl. A61H 33/00; A61G 7/057

(52) U.S. Cl. CPC A61H 33/6005; A61H 33/0095; A61M 25/0017; A61H 2033/0041; A61G 7/057

(57) ABSTRACT

This present invention relates to a wellness device in the form of a wet bed or chamber for use by bedridden individuals, preferably in a hospital, nursing or hospice care setting. The bed or chamber is comprised of a fiberglass tub filled with treated water or embryonic-like fluid in which a patient may be positioned. The bed also features several cabinets on the base to store pumps, filters, a heater, and to offer storage, and a repositionable panel for privacy. The embryonic-like fluid moisturizes the patient's skin, and maintains a desired body temperature without the worry of the patient developing bed sores. A catheter may also be attached to the patient to automatically remove bodily waste and transfer the same to a waste container for appropriate disposal.

### CROSS-REFERENCE TO RELATED APPLICATION

The present application claims priority to, and the benefit of, U.S. Provisional Application No. 62/988,457, which was filed on Mar. 12, 2020 and is incorporated herein by reference in its entirety.

### FIELD OF THE INVENTION

The present invention relates generally to the field of wellness devices. More specifically, the present invention relates to a wellness device in the form or a wet bed or chamber for hospital and/or hospice care patients. The wet bed/chamber is designed to allow the patient to lay in a pool of embryonic-like fluid, wherein the fluid functions as a way to moisturize skin and maintain a desired body temperature without the worry of developing bed sores from repeatedly laying in the same position for a prolonged period of time. The wet bed/chamber also ensures that any waste emanating from the patient is properly and efficiently filtered from the bed/chamber, thereby offering a more sanitary method of dealing with incontinence when compared to adult diapers. The wet bed/chamber also prevents the caregiver from constantly having to turn or reposition the patient, thereby saving extensive time and effort for caregivers, nurses and other medical professionals. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, hospital and/or nursing care beds are commonly used by patients in a hospital, nursing home or hospice setting, and are specifically used to cater to the needs of ill or disabled individuals and patients. Such patients are typically bedridden and incontinent individuals, who are confined to a bed and need specialized assistance in everyday activities such as hygiene, nutrition, incontinence protection and more. For example, for incontinence protection, caretakers may be responsible for changing diapers or pads of the incontinent and/or bedridden patients throughout the day and night, which is both labor intensive and time-consuming. Also, individuals wearing diapers for a prolonged period of time may suffer from Perineal dermatitis, which causes inflammation of the skin in the genital, buttock, or upper leg areas that often causes changes in the skin barrier, including redness, rash, or vesiculation, and symptoms such as pain or itching. Therefore, to minimize rashes due to wearing a diaper, the caretakers oftentimes apply various rash creams, moisturizers and more while changing the diapers. The complete process of having to frequently change diapers is tiresome and inconvenient, thereby leaving the caretakers feeling frustrated. Further, if the soiled diapers are not changed timely and frequently, the patients may be left lying in their own waste for a prolonged period of time until the diaper can be changed. This causes discomfort and shame to the bedridden patient and could also lead to infections and other complications.

Additionally, patients lying in one position for too long in a bed may develop pressure sores and other skin ailments. Injuries such as bed or pressure sores are open wounds that can become infected and cause extreme discomfort for the patient. To prevent a bedridden individual from developing bed or pressure sores, a caretaker must manually reposition the patient every few hours. Further, during each such repositioning, the caretaker must take care to ensure that the bedridden individual is clean and in good hygiene, which is both time consuming and labor intensive and therefore may be overlooked.

Therefore, there exists a long felt need in the art for a wellness device in the form of a wet bed or chamber for hospital and hospice care patients which offers a more comfortable and effective method of relaxation for the patient. There is also a long felt need in the art for a wet bed or chamber that eliminates the need for the caretaker to frequently change the diaper of an incontinent patient when the patient is utilizing the wet bed or chamber. Additionally, there is a long felt need in the art for a wet bed or chamber that ensures proper hygiene of the bedridden individual, preventing the individual from suffering from skin rashes, irritation and more, and that also minimizes the responsibility of the caretaker to frequently reposition the bedridden individual. Moreover, there is a long felt need in the art for a wet bed or chamber that allows the patient to soak in an embryonic-like fluid, wherein the embryonic-like fluid both soothes and moisturizes the skin and is continuously filtered to remove any waste emanating from the patient. Finally, there is a long felt need in the art for a wet bed or chamber that is relatively inexpensive to manufacture, and that is both safe and easy to use.

The subject matter disclosed and claimed herein, in one embodiment thereof, comprises a wellness device in the form of a wet bed or chamber designed for bedridden individuals in a hospital, nursing home or hospice care setting. The device is comprised of a housing that supports a tub portion filled with an embryonic-like (i.e., exhibits the consistency and buoyancy properties) fluid, a pump, a filter and a plurality of other components that collectively enable the patient to be suspended in the heated embryonic-like fluid to promote healing and reduce the likelihood of the patient developing pressure sores. The tub is preferably comprised of fiberglass and is configured to accommodate a patient in a supine or lateral position, with the slide cover over the fiberglass tub providing the patient with the requisite level of privacy while utilizing the device. Further, the embryonic-like fluid functions as a way to moisturize the patient's skin and maintain a desired body temperature without the worry of developing bed sores. The device further comprises a plurality of openings and related piping to collect and properly dispose of the patient's bodily waste.

In this manner, the novel wellness device of the present invention accomplishes all of the forgoing objectives, and provides a relatively comfortable, convenient and sanitary solution to provide relaxation and sleep to bedridden and incontinent individuals, without the risk of developing pressure sores. The wellness device of the present invention is also user friendly, as it does not require a caretaker to change the diapers or the position of the bedridden patients as often, thereby reducing the labor of the caretaker and saving extensive time and effort. Additionally, the wellness device of the present invention provides a sanitary environment for bedridden and incontinent patients, and prevents the patient from suffering from skin rashes and/or bed sores.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a wellness device designed for bedridden individuals in a hospital, nursing home or hospice care setting. The wellness device is comprised of a generally cuboidal-shaped housing with a plurality of built-in cabinets offering storage space for pumps, filters, and a heater, and a removably attached fiberglass tub placed along the top surface of the housing wherein the fiberglass tub is in fluid communication with an embryonic-like fluid reservoir. The fiberglass tub is configured to accommodate a patient in a supine or lateral position, and the housing further comprises a repositionable panel over the fiberglass tub to cover the body of the patient and provide privacy thereto. The embryonic-like fluid in the tub functions as a way to moisturize the skin of the patient and maintain a desired body temperature via the heater, without worry of the patient developing bed sores when using the wellness device. Further, the housing may further comprise one or more openings to accommodate a bodily waste discharge device, such as a catheter, that is in fluid communication with one or more bio-hazard waste containers.

In a further embodiment of the present invention, an incontinence controlling system for bedridden patients is disclosed and comprises a unique wet bed or chamber with a removably attached fiberglass tub placed along the top surface of the bed wherein embryonic-like fluid is poured in the fiberglass tub and a patient is laid down in the fiberglass tub in the embryonic-like fluid. Further, the wet bed or chamber is further comprised of one or more of a pump, filter, heater, and a waste discharging and collection system comprised of a plurality of pipes in fluid communication with waste collection bags, wherein the plurality of pipes, such as a catheter, channel the bodily waste from the patient to the waste collection bags hung on a stand (free standing or attached to the housing) or positioned adjacent to the wet bed/chamber. Additionally, both the stand and the wet bed/chamber may be supported on one or more wheels to make the incontinence controlling system easily portable.

In yet a further embodiment of the present invention, a method for treating incontinence and preventing bed sores in a bedridden patient is disclosed. The method comprises the initial step of supplying a housing having a fiberglass tub, one or more pumps, filters and a heater housed therein. Next, filling the fiberglass tub with an embryonic-like fluid up to a desired level, and then laying a bedridden patient in the fiberglass tub with a portion of the body covered with the embryonic-like fluid. Next, connecting a catheter to the body of the patient, wherein the catheter is in fluid communication with a waste collection bag. Finally, partially covering the fiberglass tub with a slide cover to cover the body of the patient up to the patient's neck to provide the patient with privacy, and continuously filtering and heating the embryonic-like fluid to maintain the body temperature of the patient and keep the embryonic-like fluid clean and free to bacteria, germs and the like. The embryonic-like fluid moisturizes the patient's skin, and prevents the patient from developing bed sores.

In still another embodiment of the present invention, a portable incontinence controlling chamber for use by bedridden patients is disclosed. The chamber comprises a housing that supports a fiberglass tub that is filled with treated water mixed with an antiseptic solution, a treated water reservoir to fill the fiberglass tub with treated water, a plurality of cabinets storing a heater to heat the treated water, a filter device to filter the treated water, a drain to drain the treated water after use and a repositionable panel to slide over the fiberglass tub to partially cover the patient soaking in the fiberglass tub to provide privacy thereto. The chamber may further comprise one or more waste containers in fluid communication with a catheter or other bodily fluid drainage system for discharging bodily fluids from the patient.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the wellness device of the present invention in accordance with the disclosed architecture, wherein the repositionable top panel is in a closed position;

FIG. 2 illustrates a top perspective view of one potential embodiment of the wellness device of the present invention in accordance with the disclosed architecture, wherein a bedridden patient is soaking in the fluid contained in the tub portion of the device and the repositionable top panel is in an open position;

FIG. 3 illustrates a top perspective view of one potential embodiment of the wellness device of the present invention in accordance with the disclosed architecture, wherein a bedridden patient is soaking in the fluid contained in the tub portion of the device and the repositionable top panel is in the closed position;

FIG. 4 illustrates a side perspective view of one potential embodiment of the wellness device of the present invention in accordance with the disclosed architecture; and

FIG. 5 illustrates a perspective view of one potential embodiment of the wellness device of the present invention in accordance with the disclosed architecture, wherein a bedridden patient is soaking in the fluid contained in the tub portion of the device and a second bedridden patient is laying atop of a second bed portion.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a wellness device that offers a more comfortable and effective method of relaxation for a bedridden patient, and that eliminates the need for a caretaker to frequently change the diaper of an incontinent patient when the patient is utilizing the device. Additionally, there is a long felt need in the art for a wellness device that ensures proper hygiene of the bedridden individual, prevents the individual from suffering from skin rashes, irritation, etc., and that also minimizes the responsibility of the caretaker to frequently reposition the bedridden individual to avoid the development of pressure sores. Moreover, there is a long felt need in the art for a wellness device that allows the patient to soak in an embryonic-like fluid, wherein the embryonic-like fluid both soothes and moisturizes the skin and is continuously filtered to remove any waste emanating from the patient. Finally, there is a long felt need in the art for a wet bed or chamber that is relatively inexpensive to manufacture, and that is both safe and easy to use.

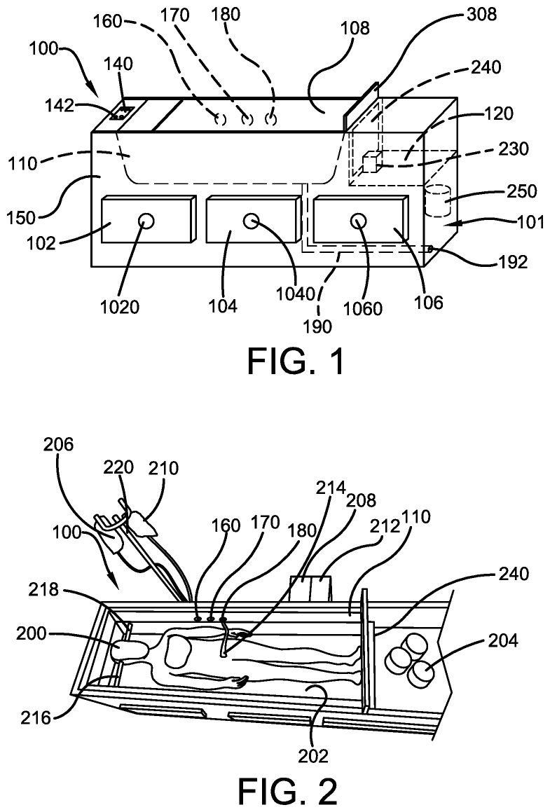

Referring initially to the drawings, FIG. 1 illustrates a perspective view of one potential embodiment of the wellness device 100 of the present invention in accordance with the disclosed architecture, wherein a repositionable top panel 108 is in a closed position. More specifically, the wellness device is designed for use by bedridden individuals in a hospital, nursing home or hospice care setting. The device 100 is comprised of a generally cuboidal-shaped housing 101 having a plurality of drawer style cabinets 102, 104, 106 positioned along a front, side or back surface, wherein the cabinets can be accessed by using the handles or knobs 1020, 1040, 1060 thereon respectively. In the embodiment shown in FIG. 1, the cabinets 102, 104, 106 are present on the front surface 150 of the housing 101, and may be used to store a pump 230, filter 240, heater 250, etc., and offer storage for any additional necessary items such as medical supplies, extra catheters 214, etc. The housing 101 can be comprised of a wood, metal or heavy-duty plastic. In one embodiment, the housing further comprises a control panel 140 for adjusting the various functions of the device 100 as explained more fully below.

A partition 308 and a fiberglass tub 110 may be positioned over top of the cabinets 102, 104, 106, as best shown in FIGS. 1 and 4. The fiberglass tub 110 is of such a length that a person can easily lie down flat in the fiberglass tub 110. The top section of the fiberglass tub 110 forms the top of the housing 101 and is generally open to allow a bedridden patient to be easily positioned in or removed from the tub 110. Nonetheless, a slidable or repositionable cover 108 may also be used to substantially cover the opening of the tub 110 when the same is not in use, or when a patient is utilizing the tub 110 to provide privacy to the patient who may be unclothed. An additional storage space 120 is also present within the housing 101 adjacent to the tub and sectioned off by partition 308. The storage space 120 may be used to store one or more of the pump 230, filter 240, heater 250, etc., or any additional and necessary items.

The cabinets 102, 104, 106 have telescopic mechanisms or rails to open and close and have depth to store bulky medicinal and personal care equipment. The rear surface (not shown), which is generally opposite and parallel to the front surface 150, preferably comprises a plurality of openings 160, 170, 180 therein for the receipt of piping and/or a catheter 214 to drain bodily waste from a patient utilizing the wellness device 100 to one or more bodily waste containers 208, 212 until the same can be properly disposed of. The openings 160, 170, 180 can be of the same or different sizes, and may also be present on any other surface of the housing 101, provided that the bedridden patient can remain in a supine position or lateral position while in the fiberglass tub 110.

The fiberglass tub 110 is also removably attached to the housing 101 and can be available in different sizes and/or configurations to satisfy the needs or preferences of the user. For example, in one embodiment, the fiberglass tub 110 is approximately seven feet long by three feet wide. The fiberglass tub 110 may be filled with an embryonic-like fluid or other medicated liquid 202 to moisturize the patient's skin and allow the patient to lie in the fluid for rest and relaxation, wherein the fluid 202 provides a degree of buoyancy to the patient and reduces the likelihood of pressure sores forming while the patient is positioned in the tub 110. The embryonic-like fluid 202 can be filled automatically through embryonic-fluid filled containers placed in the storage area 120 or can be filled manually. As best shown in FIG. 1, the tub 110 further comprises a drain line 190 in fluid communication with both the tub 110 and an outlet positioned along an outer surface of the housing 101 for draining the tub 110 of the embryonic-like or other fluid 220 when the same is no longer needed.

FIG. 2 illustrates a top perspective view of one potential embodiment of the wellness device 100 of the present invention in accordance with the disclosed architecture, wherein a bedridden patient 200 is soaking in the fluid 202 contained in the tub portion 110 of the device 100 and the repositionable top panel 108 is in an open position. More specifically, the fiberglass tub 110 has an embryonic-like fluid 202 contained therein to support the patient 200, moisturize his or her skin and provide relaxation to the patient 200. The fiberglass tub 110 may be filled with the embryonic-like or other medicated fluid 202 from one or more fluid containers 204 positioned in the housing 101, as best shown in FIG. 2, or manually by a caregiver. For example, an inlet spray might be present and in fluid communication with each of the tub 110 and the containers 204 to automatically fill the tub 110 with the embryonic-like fluid 202.

The bedridden patient 200 can remain in a supine position or in a lateral position within the tub and optional head alignment supports 216, 218 may be used to support the head of the bedridden patient 200 and keep the same above the fluid 202 level in the tub 110. In this manner, the bedridden patient 200 can easily breathe while lying in the fluid 202 in the fiberglass tub 110. A stand 220 may also be attached to the housing 101 or be free-standing and may be used to support one or more fluids or medications 206, 210 that can be intravenously fed to the patient while utilizing the device 100. Similarly, a catheter 214 may carry the patient's urine through one of the openings 160, 170, 180 to waste containers 208, 212. This offers a more sanitary method of dealing with incontinence when compared to adult diapers. Similarly, other bodily fluids may be carried out by pipes 206, 210 extending out of openings 160, 170 respectively. The bodily wastes are collected in the bodily waste containers 208, 212, which can be disposed of easily after use.

In one embodiment, a faucet (not shown) may also be present to supply the embryonic-like fluid 202 to the fiberglass tub 110. Further, as stated above, the device 100 may further comprise one or more pumps 230, filters 240 and/or heaters 250. For example, the pump could be used to fill and/or drain the tub 110, pump the fluid 202 through one or more filters 240 to cleanse the same and/or for any other fluid handling purpose associated with the device 100. The filters 240 may be used to filter out any impurities in the fluid 202 and keep the same clean and sanitary. The heater 250 may be used to maintain the fluid 202 at a comfortable temperature, similar to the body temperature of the patient 200.

The tub 110 can also be used as a bathtub for bedridden individuals to be cleaned with traditional soap and water. The tub 110 may be comprised of a fiberglass or a polyvinyl chloride (PVC) laminated material that is both somewhat flexible and adaptable to the size of the patient 200. The various pipes and drains can be easily connected to the body of the tub 110 and are commercially available. In one embodiment, a suction head (not shown) may be present along the pipes to automatically remove bodily waste into the containers 208, 212.

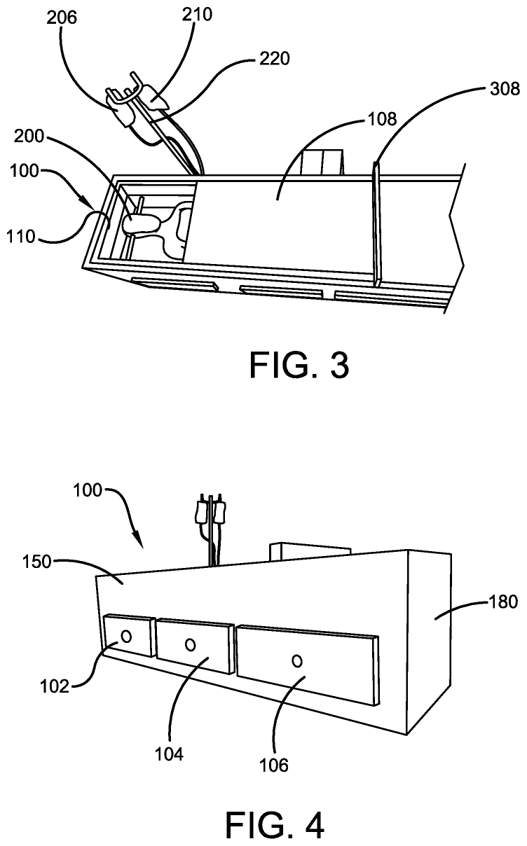

FIG. 3 illustrates a top perspective view of one potential embodiment of the wellness device 100 of the present invention in accordance with the disclosed architecture, wherein a bedridden patient 200 is soaking in the fluid 202 contained in the tub portion 110 of the device 100 and the repositionable top panel 108 is in the closed position to provide the patient 200 with a degree of privacy. More specifically, the patient 200 may lie nude in the fiberglass tub 110, and be partially covered by the sliding panel 108 which slides over the edges of the fiberglass tub 110. Notwithstanding, the repositionable panel 108 may also be hingedly attached to one or more sides of the housing 101, or can be flexible and rolled on a partition 308. The panel 108 may also have a hydraulic mechanism (not shown) for repositioning the same, but should be of a length such that it does not completely cover the top section of the fiberglass tub 110. Alternatively, stoppers may be present at a certain length of the tub 110 so that the slider 108 is prevented from completely closing on a patient 200. The slider 108 is preferably comprised of an opaque material and covers the body portion of the patient 200.

In one embodiment, the housing 101 may further comprise one or more wheels to enable a caregiver to easily reposition or transport the same. The stand 220 may also have wheels (if freestanding and not attached to the housing 101) to also make the stand 220 portable. The cabinets 102, 104, 16 may also have locks to securely store the pumps 230, filters 240, and heater 250 therein, or any other item.

FIG. 4 illustrates a side perspective view of one potential embodiment of the wellness device 100 of the present invention in accordance with the disclosed architecture. More specifically, the device 100 has the general appearance of a conventional bed with an open top surface, a front surface 150 and a pair of side surfaces. As previously stated, the front surface 150 has a plurality of cabinet drawers 102, 104, 106. In one embodiment, the dimensions of the housing 101 may be approximately seven feet in length (as measured between sides) by approximately three feet in height (as measured from a ground surface to the top surface) and approximately three and a half to four feet in width (as measured from the front surface 150 to the opposing rear surface).

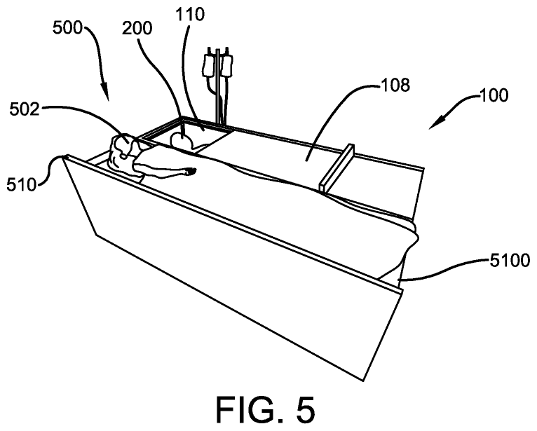

FIG. 5 illustrates a perspective view of one potential embodiment of the wellness device 500 of the present invention in accordance with the disclosed architecture, wherein a bedridden patient 200 is soaking in the fluid 202 contained in the tub portion 110 of the device 500 and a second bedridden patient 502 is laying atop of a second bed portion 510. More specifically, an extended bed 510 is present which has a surface 5100 upon which an additional user 502 can lie while waiting for treatment in the fiberglass tub 110. Simultaneously, a user 200 is getting treatment in the fiberglass tub 110 and is partially covered with the sliding panel 108 to provide privacy thereto.

As stated above, a control panel 140 may also be present on the housing 101 and includes a plurality of buttons or other controls 142 for controlling the various functions of the device 100, 500 including, without limitation, the flow, level, temperature and/or filtering of the fluid 202. The device 100, 500 of the present invention ensures that bodily waste is filtered from the tub 110, thereby offering a more sanitary method of dealing with incontinence when compared to adult diapers, and prevents a caregiver from having to constantly turn the patient 200, thereby saving extensive time and effort for nurses and other medical professionals.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “wet bed”, “wet chamber”, “nursing bed”, “chamber” and “incontinence protection bed” are interchangeable and refer to the wellness device 100 of the present invention.

Notwithstanding the forgoing, the wellness device 100 of the present invention can be of any suitable size and configuration as is known in the art without affecting the overall concept of the invention, provided that it accomplishes the above stated objectives. One of ordinary skill in the art will appreciate that the size, configuration and material of the wellness device 100 and its various components as shown in the FIGS. are for illustrative purposes only, and that many other sizes and shapes of the wellness device 100 are well within the scope of the present disclosure. Although the dimensions of the wellness device 100 and its various components are important design parameters for user convenience, the wellness device 100 and its components may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A wellness device comprising:
a housing having a top portion;
a tub at least partially filled with an embryonic-like substance;
a pump;
a filter; and
a sliding cover repositionable along the top portion.

2. The wellness device as recited in claim 1, further comprising at least one cabinet.

3. The wellness device as recited in claim 2, wherein the pump is in fluid communication with the tub.

4. The wellness device as recited in claim 3, wherein the embryonic-like substance is passed through the filter.

5. The wellness device as recited in claim 4 further comprising a catheter.

6. The wellness device as recited in claim 5 further comprising a stand.

7. The wellness device as recited in claim 6 further comprising a partition.

8. The wellness device as recited in claim 7 further comprising an alignment support.

9. The wellness device as recited in claim 5 further comprising a bodily waste container.

10. The wellness device as recited in claim 9, wherein the bodily waste container is in fluid communication with the catheter.

11. The wellness device as recited in claim 10 further comprising an extended bed portion.

12. A wellness device comprising:
a generally rectangular housing comprised of a front panel;
a tub at least partially filled with an embryonic-like substance and positioned within the housing;
a filter; and
at least one cabinet positioned along the front panel.

13. The wellness device as recited in claim 12 further comprising a pump, a drain and an outlet.

14. The wellness device as recited in claim 13, wherein the embryonic-like substance is passed through the filter.

15. The wellness device as recited in claim 14 further comprising a catheter.

16. The wellness device as recited in claim 15 further comprising a stand.

17. The wellness device as recited in claim 16 further comprising a partition.

18. The wellness device as recited in claim 17 further comprising an alignment support and a bodily waste container in fluid communication with the catheter.

19. A wellness device comprising:
a generally rectangular housing;
a tub at least partially filled with an embryonic-like substance and positioned within the housing;
a bed portion positioned within the housing adjacent to the tub;
a filter, wherein the embryonic-like substance is passed through the filter;
a pump;
at least one cabinet;
a drain in fluid communication with each of a bottom surface of the tub and an outlet; and
a catheter in fluid communication with a waste container.

20. The wellness device as recited in claim 19 further comprising a heater.
